(19) United States
(12) Patent Application Publication
(10) Pub. No.: US 2007/0118200 A1
(43) Pub. Date: May 24, 2007

(54) Bifurcation stent delivery system

(75) Inventors: Jan Weber, Maple Grove, MN (US); Karl A. Jagger, Deephaven, MN (US); Tracee Eidenschink, Wayzata, MN (US)

Correspondence Address: MERCHANT & GOULD PC, P.O. BOX 2903, MINNEAPOLIS, MN, 55402-0903, US

(73) Assignee: Boston Scientific Scimed, Inc., Maple Grove, MN (US)

(21) Appl. No.: 11/282,896

(22) Filed: Nov. 18, 2005

(51) Int. Cl. A61F 2/06

(52) U.S. Cl. 623/1.11

(57) ABSTRACT

A catheter assembly includes a catheter, a balloon, a plurality of guidewire housings, and a stent. The stent is disposed about the balloon. At least a portion of each of the guidewire housings is positioned between the stent and the balloon.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

In some embodiments this invention relates to implantable medical devices, their manufacture, and methods of use. Some embodiments are directed to delivery systems, such as catheter systems of all types, which are utilized in the delivery of such devices.

2. Description of the Related Art

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents and similar devices such as stent, stent-grafts, expandable frameworks, and similar implantable medical devices, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. Stents may be used to reinforce body vessels and to prevent restenosis following angioplasty in the vascular system. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

The invention contemplates a new apparatus and method that simplifies placement of a stent at the bifurcation of a vessel. The invention results in a reduced stent delivery system profile. The present system may improve trackability of the stent delivery system.

At least one of the embodiments of the present invention includes a medical device with a balloon catheter shaft such as described in U.S. patent application Ser. No. 10/747,546, filed Dec. 29, 2003 entitled Rotating Balloon Expandable Sheath Bifurcation Delivery System and U.S. patent application Ser. No. 10/226,362, filed Aug. 22, 2002 entitled Rotating Stent Delivery System For Side Branch Access And Protection And Method Of Using Same, the entire content of both incorporated herein by reference.

In at least one embodiment there are two guidewire housings positioned on the exterior of the balloon catheter. The guidewire housings can be formed in a number of different shapes, all of which are constructed and arranged to allow passage of a guidewire through the housing. For example, in a preferred embodiment, a guidewire housing can be substantially cylindrical, like a tubular sheath. Or, a guidewire housing can be formed as rail. In other embodiments, the guidewire housing can be crescent-shaped. Alternatively, the guidewire housing could be designed such that a cross-section of the guidewire housing is semi-circular.

Rotating a stent delivery system delivered to a site within the body is difficult. In order to rotate the stent delivery system, the torque applied must be large enough to exceed the torsional stiffness of the stent delivery system. The torque with respect to the stent delivery system is maximized if the radial distance between the guidewire housings, and thus the guidewires, is maximized.

In a preferred embodiment, the radial distance between the guidewire housings, and necessarily the guidewires themselves, is maximized. Specifically, in this embodiment, the two guidewire housings are positioned on substantially opposite portions of the balloon. Thus, in this embodiment, the torque about the stent delivery system is maximized.

In at least one embodiment, the guidewire housings are not attached to the balloon. Rather, a stent is disposed about the balloon, with the guidewire housings loosely placed in between the stent and balloon, and then the stent is crimped over the guidewire housings, securingly engaging the guidewire housings to the balloon and stent. In some embodiments, the guidewire housing is, however, attached to the proximal end of the catheter shaft. By attaching the guidewire housing to the catheter shaft, and only crimping the guidewire housing underneath the stent, the stent delivery system can be maneuvered within the lumen of a vessel.

In another advantageous embodiment, the catheter shaft includes an opening that allows inflation fluid to flow into the balloon, thereby causing the balloon to inflate.

A primary feature of some embodiments is the inclusion of a support tube within the catheter shaft. In a preferred embodiment, the support tube is not connected to the catheter shaft. Instead, the catheter shaft is disposed loosely about the support tube. By including a support tube within the catheter shaft, the catheter shaft will rotate around the support tube when the stent delivery system is rotated, thereby preventing the catheter shaft from forming a kink. As the catheter shaft forms the path in which any inflation fluid is delivered to the balloon, any kinks within the catheter shaft would detrimentally interfere with the fluid delivery. In a preferred embodiment, the support tube is hollow, allowing inflation fluid to flow through the support tube, as well as around it within the catheter shaft. Other embodiments include a support tube that is solid rather than hollow.

In a preferred embodiment, the support tube is formed in the shape of a spiral. It is also envisioned that in some embodiments the support tube is substantially cylindrical. The support tube could be formed in a number of other shapes, such as with a square or rectangular cross section.

These and other embodiments which characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there is illustrated and described embodiments of the invention.

### BRIEF DESCRIPTION OF THE DRAWINGS

FIG. 1 is a side view of an embodiment of the invention, comprising a stent, balloon catheter, and guidewire housings.

FIG. 2 is a perspective view of an embodiment of a guidewire housing with circular cross-section.

FIG. 3 is a perspective view of an embodiment of a guidewire housing with semi-circular cross-section.

FIG. 4 is a perspective view of an embodiment of a guidewire housing with crescent-shaped cross-section.

FIG. 5 is a perspective view of an embodiment of a guidewire housing formed as a rail wherein the lumen is partially formed by the balloon.

FIG. 6 is a transverse cross-sectional view of the embodiment depicted in FIG. 1.

FIG. 7a is a side view of an embodiment of the invention, with guidewire housings engaged to the proximal end of a catheter shaft.

FIG. 7b is a side perspective view of a stent wherein a side branch opening is shown formed from the enlargement of a cell opening in the stent wall.

FIG. 7c is a cross-sectional view of the stent of FIG. 7b.

FIG. 7d is a side view of a stent wherein the stent has been delivered from the catheter assembly, by balloon expansion and the assembly subsequently withdrawn from the vessel(s).

FIG. 8 is a side view of an embodiment of the invention, shown with a spiral support tube.

FIG. 9 is a side view of an embodiment of the invention, shown with a support tube of substantially elongate shape.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

Depicted in the figures are various aspects of the invention. Elements depicted in one figure may be combined with, and/or substituted for, elements depicted in another figure as desired.

Referring now to the drawings, wherein the showings are for the purposes of illustrating the preferred embodiments of the invention and not for purposes of limiting same, FIG. 1 shows a stent delivery system or assembly 5. Assembly 5 shows a catheter 10 comprising catheter shaft 15. Disposed about catheter shaft 15 is balloon 20. Disposed about balloon 20 is stent 25. There is a primary guidewire housing 30 and a secondary guidewire housing 35 positioned on the exterior of balloon 20. The primary guidewire housing 30 and secondary guidewire housing 35 can be formed in a number of different shapes, all of which are constructed and arranged to allow passage of a guidewire through the guidewire housing. Primary guidewire 40 passes through primary guidewire housing 30 and secondary guidewire 45 passes through secondary guidewire housing 35. Catheter shaft 15 also includes an opening 50, positioned underneath balloon 20. Opening 50 allows an inflation fluid (not shown) to be injected into balloon 20 through catheter shaft 15.

In some embodiments, the secondary guidewire housing may not be external to the balloon, as shown in FIG. 1. Instead, the secondary guidewire housing may be incorporated with the balloon. For example, the secondary guidewire may be placed such that the balloon is folded about the secondary guidewire during manufacture. The balloon itself would thereby define a secondary guidewire lumen. In another example, the balloon material could be manufactured such that a cavity, defining a secondary guidewire lumen, is incorporated within the balloon wall thickness, thereby allowing a secondary guidewire to be inserted therethrough.

In some embodiments the stent, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In some embodiments the at least a portion of the stent is configured to include one or more mechanisms for the delivery of a therapeutic agent. The agent can be in the form of a coating or other layer (or layers) of material placed on a surface region of the stent, which is adapted to be released at the site of the stent's implantation or areas adjacent thereto. In some embodiments, a therapeutic agent may be delivered from the catheter and/or stent via a lumen, opening, or other delivery mechanism.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

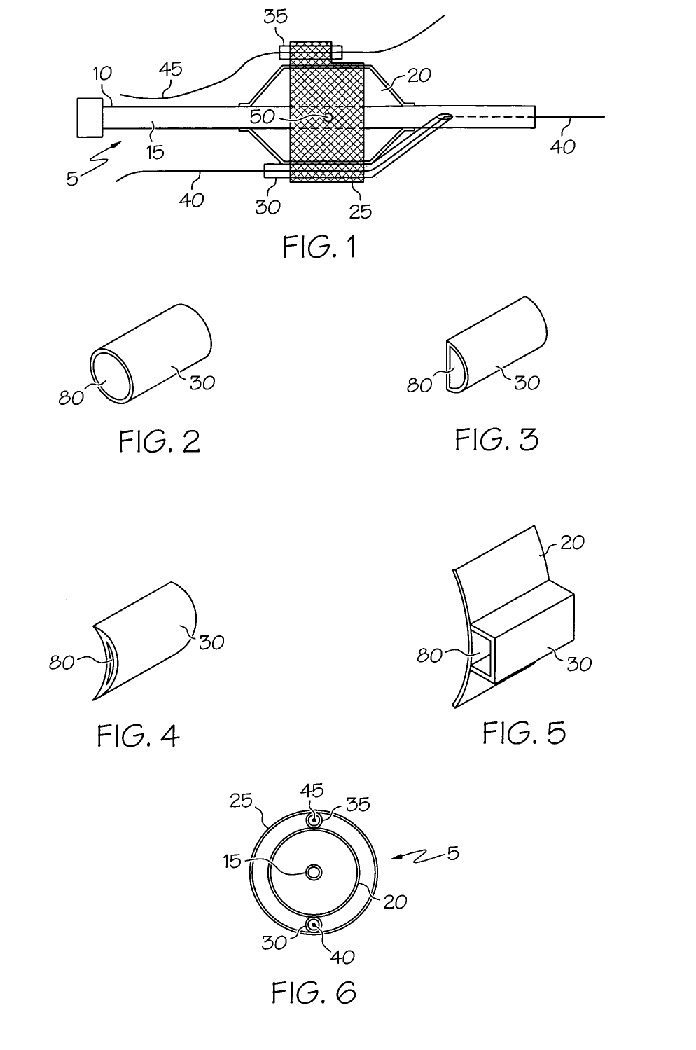

FIG. 2 shows a preferred embodiment of primary guidewire housing 30, defining guidewire lumen 80, in substantially cylindrical form. In another embodiment, FIG. 3 shows the primary guidewire housing 30, defining guidewire lumen 80, with a design that has a semi-circular cross-section. FIG. 4 shows the primary guidewire housing 30, defining guidewire lumen 80, with a design that has a crescent-shaped cross-section. FIG. 5 shows the primary guidewire housing 30 formed as a rail, such that the guidewire lumen 80 is at least partially defined by the external surface of the balloon 20. Although reference number 30 has been used in FIGS. 2 through 5, it should be pointed out that each of these designs applies to secondary guidewire housing 35, or any other guidewire housing, as well.

FIG. 6 depicts a transverse cross-section of the assembly 5 of FIG. 1. Primary guidewire housing 30 and secondary guidewire housing 35 are crimped between balloon 20 and stent 25. In this embodiment, guidewire housings 30 and 35 are not attached to balloon 20. Rather, a stent 25 is disposed about balloon 20, with guidewire housings 30 and 35 placed in between stent 25 and balloon 20, and then stent 25 is crimped over guidewire housings 30 and 35, securingly engaging guidewire housings 30 and 35 to balloon 20 and stent 25.

While advancing assembly 5 to the bifurcation site, assembly 5 is rotatable in order to align the stent with the bifurcation. FIG. 6 depicts a preferred embodiment, wherein the radial distance between the guidewire housings 30 and 35, and necessarily guidewires 40 and 45, is maximized. Specifically, as shown in FIG. 6, guidewire housings 30 and 35 are positioned on substantially opposite portions of balloon 20. Thus, in this embodiment, the torque about the stent delivery system is maximized. Once assembly 5 is delivered to the bifurcation site, balloon 20 is expanded which, as a result, will expand stent 25.

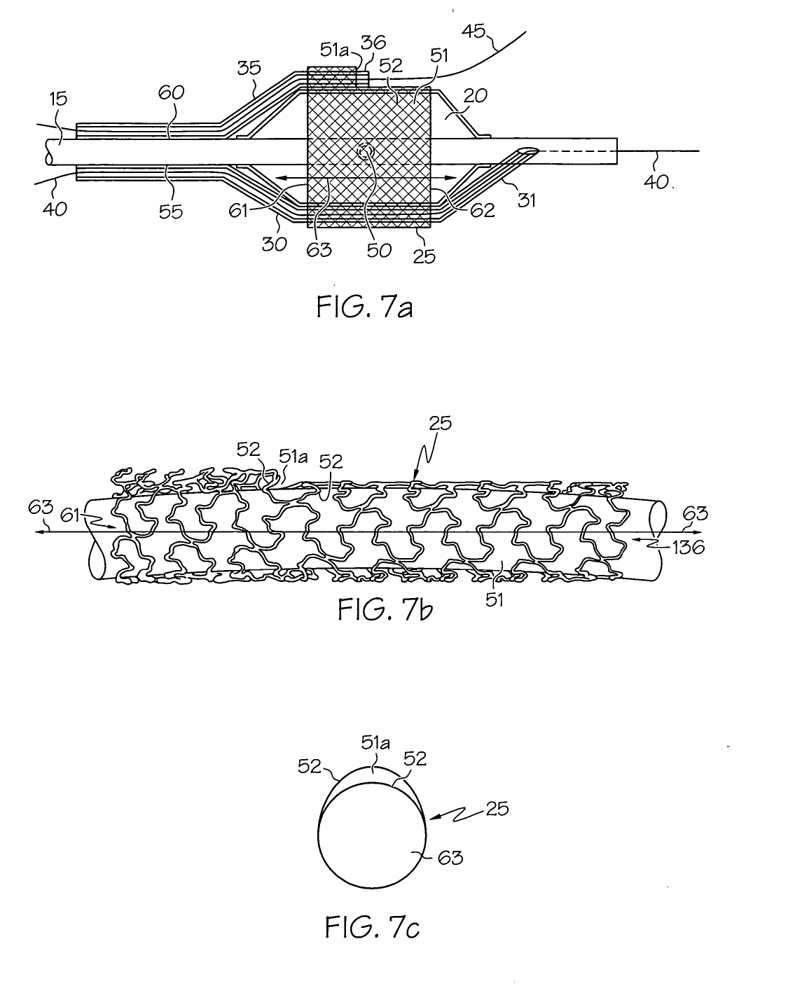

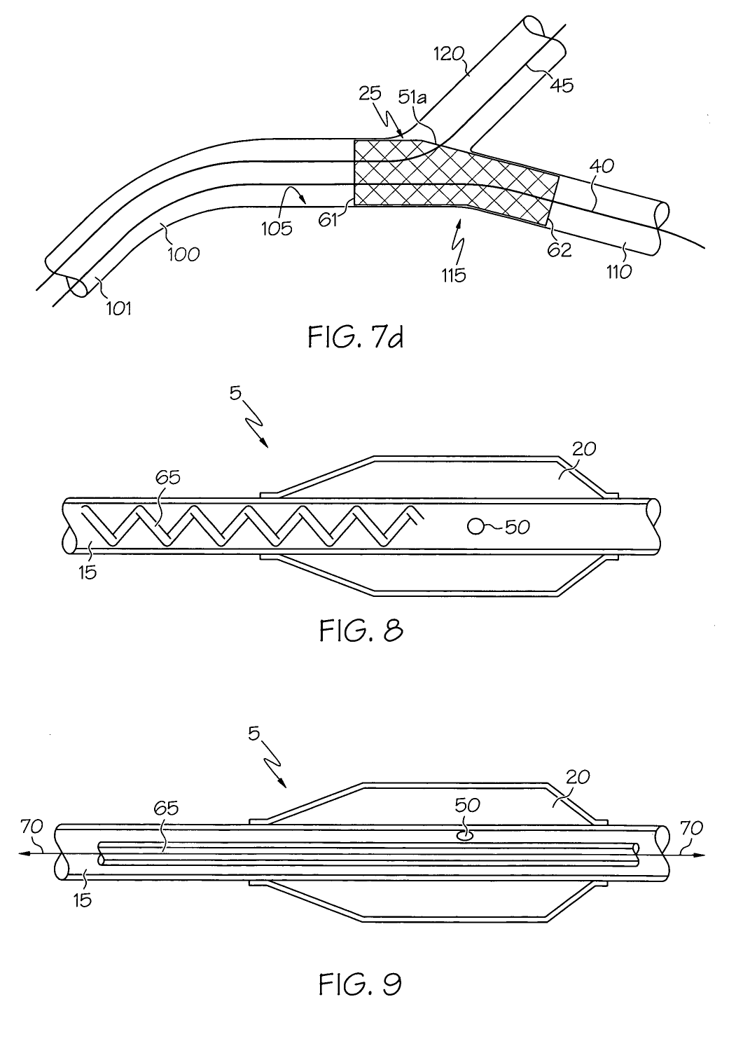

Referring now to FIG. 7a, the guidewire housings (30 and 35) are engaged to the proximal end of catheter shaft 15. Primary guidewire housing 30, having distal end 31, is engaged to catheter shaft 15 at engagement region 55. Likewise, guidewire housing 35, having distal end 36, is engaged to catheter shaft 15 at engagement region 60. By fixedly engaging guidewire housings 30 and 35 to catheter shaft 15, the trackability of stent delivery system is improved, allowing the stent delivery system to be maneuvered within the lumen of a vessel. The guidewire housings can be fixedly engaged to the catheter shaft by a number of methods, including chemical welding, heat welding, adhesives, as well as mechanical engagement. Furthermore, catheter shaft 15 includes an opening 50 that allows inflation fluid (not shown) to flow into balloon 20, thereby causing balloon 20 to inflate.

Referring to FIGS. 7a and 7b, stent 25 may be at least partially constructed of a plurality of interconnected struts, connectors, or members 52. The stent 25 defines a proximal opening 61, a distal opening 62, and a flow path 63 therebetween. The cell openings 51 are in fluid communication with the flow path 63.

When the secondary guidewire 45 and/or the secondary guidewire housing 35 is threaded through one of cell openings 51 when the stent 25 is positioned onto balloon 20, such as shown in FIG. 7a, the members 52 that define a selected cell opening 51a, may bend or flex. This bending or flexing of members 52 may result in an expansion of the shape of cell opening 51a, relative to other cell openings 51.

Referring now to FIGS. 7a and 7b, the modified cell opening 51a, hereinafter referred to as secondary opening 51a, is positioned on the stent 25 between the proximal opening 61 and the distal opening 62. The manner in which the secondary opening 51a, the members 52 adjacent thereto, and to an extent the stent 25 itself, are expanded relative to other cell openings 51 by the position of the secondary guidewire and/or secondary guidewire housing is depicted in FIGS. 7b and 7c.

It should be noted that when the stent 25 is placed on the balloon 20 in the manner described above, there is minimal flexing of members 52 and therefore substantially no expansion of cell opening 51a relative to other cell openings 51. Furthermore, the expansion of cell opening 51a, relative to other cell openings 51 is provided only to allow sliding passage of the secondary guidewire 45, and if desired, a distal portion 36 of the secondary guidewire housing 35, through the secondary opening 51a. Therefore, the actual size of the secondary opening 51a may be substantially similar to, or only marginally different from, that of the surrounding cell openings 51.

FIG. 7d shows stent 25, with proximal end 61 and distal end 62, positioned within body lumen 100 of a vessel 101, defined by vessel wall 105, at bifurcation site 115. Primary guidewire 40 extends through distal end 62 along first branch 110. Secondary guidewire 45 extends through secondary opening 51a along second branch 120.

An advantageous feature of some embodiments is shown in FIG. 8. Support tube 65 is included within catheter shaft 15. In a preferred embodiment, support tube 65 is not connected to catheter shaft 15. Instead, catheter shaft 15 is disposed loosely about support tube 65. Including support tube 65 within catheter shaft 15 allows catheter shaft 15 to rotate around support tube 65 when the assembly 5 is rotated, thereby preventing any kinks from forming in catheter shaft 15. As catheter shaft 15 forms the path in which any inflation fluid (not shown) is delivered to balloon 20 through opening 50, any kinks within catheter shaft 15 would detrimentally interfere with fluid delivery. In a preferred embodiment, support tube 65 is hollow, allowing inflation fluid to flow through support tube 65, as well as around it within catheter shaft 15. Other embodiments include a support tube 65 that is solid rather than hollow.

FIG. 8 depicts a preferred embodiment of support tube 65, formed in the shape of a spiral. It is also envisioned that in some embodiments the support tube 65 is a substantially elongate shape which extends substantially parallel to the longitudinal axis 70 of catheter shaft 15, as shown in FIG. 9. The support tube 65 could be formed in a number of other shapes, such as with star-shaped, square, or rectangular cross-sections.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

1. A catheter assembly comprising:
a catheter, the catheter comprising a catheter shaft and a balloon, the catheter shaft having a longitudinal axis therethrough, the catheter shaft defining an inflation lumen, the inflation lumen in fluid communication with the balloon, the balloon being expandable from and between a reduced diameter configuration and an expanded diameter configuration, the balloon comprising an external surface;
a stent, the stent being expandable from a reduced stent state to an expanded stent state, in the reduced stent state the stent being disposed about the balloon;
a first guidewire housing, the first guidewire housing defining a first guidewire lumen for passage of a first guidewire therethrough, at least a portion of the first guidewire housing positioned between the stent and the balloon; and
a second guidewire housing, the second guidewire housing defining a second guidewire lumen for passage of a second guidewire therethrough, at least a portion of the second guidewire housing positioned between the stent and the balloon.

2. The catheter assembly of claim 1 wherein the first guidewire housing and second guidewire housing being positioned on substantially opposite portions of the balloon.

3. The catheter assembly of claim 1 wherein at least one of the first guidewire housing and the second guidewire housing is engaged to the catheter through an engagement mechanism.

4. The catheter assembly of claim 3 wherein the engagement mechanism comprises crimping the at least a portion of the first guidewire housing and the at least a portion of the second guidewire housing underneath the stent to the balloon.

5. The catheter assembly of claim 4 wherein at least one of the first guidewire housing and the second guidewire housing is fixedly engaged to the catheter shaft.

6. The catheter assembly of claim 1 wherein the catheter shaft defines an opening, the opening in fluid communication with the inflation lumen, wherein the balloon is disposed about the opening.

7. The catheter assembly of claim 1 wherein at least one of the first guidewire housing and second guidewire housing are substantially round in cross-sectional shape.

8. The catheter assembly of claim 1 wherein at least one of the first guidewire lumen and second guidewire lumen are at least partially defined by the external surface of the balloon.

9. The catheter assembly of claim 1 wherein at least one of the first guidewire housing and second guidewire housing are substantially crescent-shaped in cross section.

10. The catheter assembly of claim 1 wherein the catheter shaft is disposed about a support tube, the support tube being independently moveable within the inflation lumen.

11. The catheter assembly of claim 10 wherein the support tube comprises a substantially elongate shape which extends substantially parallel to the longitudinal axis of the catheter shaft.

12. The catheter assembly of claim 11 wherein the support tube comprises a substantially spiral shape.

13. The catheter assembly of claim 11 wherein the support tube is substantially curvilinear.

14. The stent of claim 1 wherein the stent defines a proximal end, a distal end, and a flow path therebetween, the stent comprising members, the members defining cell openings, the cell openings in fluid communication with the flow path, at least one cell opening having a shape different than that of adjacent cell openings, the at least one cell opening positioned between the proximal end and the distal end.

15. The stent of claim 14 wherein the first guidewire extends through and beyond the distal end, the second guidewire extending through and beyond the at least one cell opening.

16. The stent of claim 14 wherein the second guidewire housing extends distally beyond the at least one cell opening.

17. The stent of claim 14 wherein the first guidewire housing extends beyond the distal end.

18. A catheter assembly comprising:
a catheter, the catheter comprising a catheter shaft and a balloon, the catheter shaft defining an inflation lumen and an opening, the opening in fluid communication with the inflation lumen, the balloon disposed about the opening, the balloon being expandable from and between a reduced diameter configuration and an expanded diameter configuration;
a stent, the stent being expandable from a reduced stent state to an expanded stent state, in the reduced stent state the stent being disposed about the balloon;
a first guidewire housing, the first guidewire housing defining a first guidewire lumen for passage of a first guidewire therethrough, at least a portion of the first guidewire housing positioned between the stent and the balloon;
a second guidewire housing, the second guidewire housing defining a second guidewire lumen for passage of a second guidewire therethrough, at least a portion of the second guidewire housing positioned between the stent and the balloon, the first guidewire housing and second guidewire housing being positioned on substantially opposite portions of the balloon;
a support tube, the catheter shaft being disposed about the support tube, the support tube being independently moveable within the inflation lumen, the support tube comprising a substantially spiral shape.

19. A method of placing a stent at a bifurcation comprising the steps of:
advancing a first guidewire through a body lumen to a first branch of a vessel bifurcation;
advancing a second guidewire through a body lumen to a second branch of a vessel bifurcation;
advancing a first catheter assembly to the vessel bifurcation along the first guidewire and a second guidewire, the first catheter assembly comprising:
a balloon disposed about at least a portion of the catheter assembly;
a first guidewire housing engaged to the external surface of the balloon, the first guidewire housing being disposed about the first guidewire;
a second guidewire housing engaged to the external surface of the balloon, the second guidewire housing being disposed about the second guidewire, the first guidewire housing and second guidewire housing being positioned on substantially opposite portions of the balloon;
a stent, the stent being disposed about at least a portion of the balloon, the stent being further disposed about at least a portion of each of the first guidewire housing and the second guidewire housing, the first guidewire passing through the region defined by the first guidewire housing, the second guidewire passing through the region defined by the second guidewire housing; and
expanding the balloon to expand the stent.

20. The method of claim 19 further comprising the steps of:
retracting the first guidewire from the body lumen;
advancing a third guidewire through a body lumen to the first branch of the vessel bifurcation;
advancing a second catheter assembly, through the expanded stent to the first branch of the vessel bifurcation along the third guidewire, the second catheter assembly comprising:
a balloon disposed about at least a portion of the second catheter assembly;
a guidewire housing engaged to the external surface of the balloon, the guidewire housing being disposed about the third guidewire;
a stent, the stent being disposed about at least a portion of the balloon, the stent being further disposed about at least a portion of the guidewire housing, the third guidewire passing through the region defined by the guidewire housing; and
expanding the balloon to expand the stent.
